(19) United States
(12) Patent Application Publication
(10) Pub. No.: US 2026/0216039 A1
(43) Pub. Date: Jul. 30, 2026

(54) SKINCARE PRODUCTS CONTAINING DUAL-ACTION PEPTIDE DELIVERY SYSTEM

(71) Applicant: Nocturnal, Inc., Los Angeles, CA (US)

(72) Inventors: Katey Hassan, Hastings On Hudson, NY (US); Daniel Kiyoi, Los Angeles, CA (US)

(21) Appl. No.: 19/459,472

(22) Filed: Jan. 26, 2026

Related U.S. Application Data
(60) Provisional application No. 63/749,391, filed on Jan. 24, 2025.

(51) Int. Cl. A61K 8/64; A61K 8/11; A61K 8/19; A61K 8/67; A61K 8/9794; A61Q 19/00

(52) U.S. Cl. CPC A61K 8/64; A61K 8/11; A61K 8/19; A61K 8/675; A61K 8/9794; A61Q 19/00

(57) ABSTRACT

A skincare composition formulated to enhance skin repair and regeneration, particularly during periods of rest or reduced recovery time. The composition may include a dual-action delivery system including a time-release delivery vehicle and a targeted delivery vehicle. In some embodiments, the time-release delivery vehicle may include a core-shell particle in which a signal peptide may be encapsulated by a gold shell to provide controlled release. The targeted delivery vehicle may be capable of selective association with specific skin cells. The composition may further include a dual peptide system comprising at least one signal peptide, associated with the time-release delivery vehicle, and at least one carrier peptide, associated with the targeted delivery vehicle.

### CROSS-REFERENCE TO RELATED APPLICATIONS

This application claims the benefit of priority to U.S. Provisional Patent Application No. 63/749,391, filed Jan. 24, 2025, and entitled “Skincare Products Containing Dual-Action Peptide Delivery System,” the entire contents of which are hereby incorporated by reference in their entirety for all purposes.

### BACKGROUND

The skin, as the largest organ of the human body, is continuously subjected to environmental stressors, including physical injury, oxidative damage, and the effects of ultraviolet (UV) radiation (known as “photoaging”), all of which contribute to the aging process and the overall degradation of skin health. Such degradation may manifest as the appearance of fine lines, deep wrinkles, uneven skin pigmentation (such as age spots or lentigines), thinning of the epidermis, and an overall dull and uneven skin texture. The skin's natural defense mechanisms, including antioxidant enzymes and DNA repair processes, also become less effective over time, allowing photoaging to progress more rapidly with continued UV exposure. The resulting the degradation of collagen fibers may further contribute to the loss of skin firmness, making the skin more susceptible to sagging and the formation of deep folds.

During the natural circadian rhythm, particularly during the sleep phase, the body engages in a variety of repair and regeneration mechanisms that are vital to maintaining skin homeostasis, promoting cellular recovery, and counteracting the effects of daily environmental exposures. During deep sleep, the body increases the production of growth factors, hormones, and cytokines, which play key roles in tissue regeneration and repair. Specifically, the secretion of growth hormone (GH) during sleep has been found to stimulate cellular proliferation, collagen synthesis, and the repair of damaged tissues, all of which are crucial for maintaining skin structure and function.

At the cellular level, sleep may induce the activation of key repair mechanisms, including the restoration of the skin's barrier function and the repair of DNA damage caused by oxidative stress and UV exposure. The skin's epidermal layer, particularly keratinocytes, undergoes increased turnover during sleep, which aids in the replacement of damaged or senescent cells. Furthermore, during the nighttime resting phase, the skin's antioxidant defense system is upregulated, leading to an enhanced ability to neutralize reactive oxygen species (ROS) and mitigate the effects of oxidative damage that accumulate during the day.

Additionally, during sleep, the process of autophagy is activated in skin cells. Autophagy, the cellular process by which damaged organelles, misfolded proteins, and other cellular debris are removed and recycled, is essential for maintaining skin health. This process helps to clear damaged components that could otherwise impair skin function or accelerate the aging process. The nightly repair process is further supported by the enhanced synthesis of extracellular matrix (ECM) proteins, such as collagen and elastin, which contribute to the skin's structural integrity and its ability to resist environmental insults.

Sleep also plays a significant role in immune function, as the body enters a more restorative phase that supports the activation of immune cells, such as macrophages, which aid in the clearance of pathogens, debris, and damaged cells from the skin. This immune response ensures the efficient resolution of inflammation and the restoration of skin health.

Thus, during sleep, the skin undergoes a coordinated series of repair mechanisms that are essential for repairing damage, restoring homeostasis, and promoting the long-term health and appearance of the skin. These processes are vital for mitigating the visible effects of aging, environmental stressors, and daily wear and tear on the skin, underscoring the importance of sleep for maintaining skin vitality and function.

Despite the importance of sleep as a skin repair mechanism, sleep interruption or deprivation is a common problem due in modern society, due to health and/or lifestyle challenges such as stress, parenthood, shift work, travel across time zones, and the like. Sleep deprivation, defined as insufficient sleep over an extended period, or sleep interruption, wherein the natural sleep cycle is disrupted, interferes with the body's ability to perform its natural repair processes. As a result, there is a pressing need for effective solutions that can mitigate the negative consequences of sleep deprivation on the skin and enhance and promote the body's natural skin repair processes in less time.

### BRIEF SUMMARY

Aspects of the present disclosure provide skincare compositions capable of enhancing and promoting skin repair mechanisms during sleep. The disclosed skincare compositions contain a dual-action peptide delivery system including a time-release vehicle and a targeted vehicle. In some aspects, the disclosed compositions may additionally include an oat lipid composition, a niacinamide, and an antioxidant.

According to some aspects, the disclosed skincare composition can include: a core-shell particle having a core comprising a signal peptide, the core encapsulated by a shell formed of gold; a carrier peptide comprising a metal-binding peptide and a lipid-modified peptide; a niacinamide; and an oat lipid composition comprising at least one of an oat extract, an oat oil, or an oat flour; wherein a weight ratio of the signal peptide to the carrier peptide is within a range of 0.05:1 to 1:1, and wherein a weight ratio of the metal-binding peptide to the lipid-modified peptide is within a range of 40:60 to 60:40.

In some instances, the signal peptide may comprise one or more of a palmitoyl pentapeptide-3, a pentapeptide-18, a palmitoyl pentapeptide-12, a acetyl pentapeptide-1, a palmitoyl tetrapeptide-7, a palmitoyl tripeptide-1, or a hexapeptide-9. In some cases, the signal peptide comprises a palmitoyl pentapeptide-4. The signal peptide may be included in the disclosed skincare composition in an amount of from about 0.00001 wt % to about 0.00005 wt %, based on a total weight of the skincare composition.

In some instances, the gold is in a form of a nanoparticle having a particle size of no greater than about 200 nm. In some cases, the gold is in a form of a nanoparticle having a D50 particle size of no greater than about 100 nm.

In some instances, the metal-binding peptide comprises a copper-binding peptide. In some cases, the metal-binding peptide comprises a copper heptapeptide-14 pantothenate. In some instances, the lipid-modified peptide comprises heptapeptide-15 palmitate. In some cases, each of the metal-binding peptide and the lipid-modified peptide comprises a heptapeptide.

In some instances, the carrier peptide comprises or is associated with a targeted delivery vehicle. In some cases, the targeted delivery vehicle comprises at least one of lipid moiety or a metal-binding ligand. The carrier peptide may be included in the disclosed skincare composition in an amount of 0.00005 wt % to about 0.0002 wt %, based on a total weight of the skincare composition.

In some instances, the oat lipid composition comprises Avena sativa kernel extract, Avena sativa kernel oil, and Avena sativa kernel flour. The oat lipid composition may be included in an amount within a range of about 1.0 wt % to about 4.0 wt %, based on a total weight of the skincare composition.

In some instances, the niacinamide is included in the disclosed skincare composition in an amount of about 1.0 wt % to about 4.0 wt %, based on a total weight of the skincare composition. In some instances, the skincare composition further includes an evening primrose oil, such as in an amount within a range of about 0.5 wt % to about 1.0 wt %, based on a total weight of the skincare composition.

In some instances, the skincare composition is an aqueous composition. In some cases, the skincare composition has a pH within a range of about 5.0 to about 6.0. In some cases, the skincare composition has a viscosity within a range of about 8,000 cps to about 11,000 cps at 25° C.

In some aspects, the present disclosure includes a method of treating or improving a cosmetic skin condition, the method comprising: applying an effective amount of the skincare composition of claim 1 to a treatment area two times per day, repeating the daily application for a time period of at least 4 weeks.

In some instances, the time period is at least 6 weeks. In some cases, the time period is no greater than 2 months, no greater than 4 months, no greater than 6 months, or no greater than a year. In some instances, the twice daily application of an effective amount of the skincare composition comprises applying an effective amount once in a morning and once in an evening.

According to some embodiments, the repetition of the daily application for the time period of 4 weeks produces at least an 11% improvement in fine lines and/or wrinkles within the treatment area in comparison to a baseline measurement taken before commencing treatment (such as on the same day or within the same week as commencement of treatment), such as an improvement within a range of 11% to 30%, within a range of 11% to 15%, within a range of 11% to 12%, or any combination therein. In some instances, the repetition of the daily application for the time period of 6 weeks produces at least an 15% improvement in fine lines and/or wrinkles within the treatment area in comparison to a baseline measurement taken before commencing treatment (such as on the same day or within the same week as commencement of treatment), such as an improvement within a range of 15% to 50%, within a range of 15% to 40%, within a range of 15% to 30%, within a range of 15% to 20%, or any combination therein. In some cases, the improvement is based on a mean area score generated by computer assisted image analysis of a digital image of the treatment area.

According to some embodiments, the repetition of the daily application for the time period of 4 weeks produces at least an 11% improvement in skin firmness within the treatment area in comparison to a baseline measurement taken before commencing treatment (such as on the same day or within the same week as commencement of treatment), such as an improvement within a range of 11% to 40%, within a range of 11% to 30%, within a range of 11% to 20%, within a range of 11% to 15%, or any combination therein. In some instances, the repetition of the daily application for the time period of 6 weeks produces at least a 40% improvement in skin firmness within the treatment area in comparison to a baseline measurement taken before commencing treatment (such as on the same day or within the same week as commencement of treatment), such as an improvement within a range of 40% to 70%, within a range of 40% to 60%, within a range of 40% to 50%, within a range of 40% to 45%, or any combination therein. In some cases, the improvement is based on a measurement by a suction-based skin measurement device such as a Cutometer.

According to some embodiments, the repetition of the daily application for the time period of 4 weeks produces at least an 20% improvement in skin moisture within the treatment area in comparison to a baseline measurement taken before commencing treatment (such as on the same day or within the same week as commencement of treatment), such as an improvement within a range of 20% to 50%, within a range of 20% to 40%, within a range of 20% to 30%, within a range of 20% to 25%, or any combination therein. In some instances, the repetition of the daily application for the time period of 6 weeks produces at least a 19% improvement in skin firmness within the treatment area in comparison to a baseline measurement taken before commencing treatment (such as on the same day or within the same week as commencement of treatment), such as an improvement within a range of 19% to 50%, within a range of 19% to 40%, within a range of 19% to 30%, within a range of 19% to 25%, or any combination therein. In some cases, the improvement is based on a measurement by a capacitance-based skin hydration measurement device such as a Corneometer.

According to some aspects, the present disclosure also provides a skincare composition capable of producing any of the results disclosed herein (e.g., above).

According to some aspects, the present disclosure further provides a skincare composition, comprising: a core-shell particle having a core comprising a signal peptide, the core encapsulated by a shell formed of gold, a carrier peptide comprising at least one of a metal-binding peptide and a lipid-modified peptide; a niacinamide, and an oat lipid composition.

According to some aspects, the present disclosure also provides a skincare composition, comprising: a delivery system comprising a time-release delivery vehicle and a targeted delivery vehicle; a dual peptide system comprising a signal peptide and a carrier peptide; a niacinamide, and an oat lipid composition.

According to some aspects, the present disclose further provides a skincare composition, comprising: a colloidal gold present in an amount within a range of 0.000005 wt % to 0.00003 wt %, based on a total weight of the skincare composition; a signal peptide present in an amount within a range of 0.00001 wt % to 0.00005 wt %, based on the total weight of the skincare composition; a metal-binding peptide present in an amount within a range of 0.00005 wt % to 0.0002 wt %, based on the total weight of the skincare composition; a lipid-modified peptide present in an amount within a range of 0.00005 wt % to 0.0002 wt %, based on the total weight of the skincare composition; niacinamide present in an amount within a range of 1.0 wt % to 4.0 wt %, based on the total weight of the skincare composition; and an oat lipid composition present in an amount within a range of 1.0 wt % to 4.0 wt %, based on the total weight of the skincare composition.

A better understanding of the nature and advantages of embodiments of the present invention may be gained with reference to the following detailed description and the accompanying drawings.

### Terms and Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, values may be expressed in range format for convenience and brevity. Such ranges are not intended to be limiting and are deemed to expressly disclose all subranges and individual values encompassed therein. For example, a range of 1 to 6 includes subranges such as 1 to 3, 2 to 4, and 3 to 6, as well as individual values including 1, 2, 3, 4, 5, and 6, regardless of the breadth of the range.

As used in the specification and claims, the singular forms “a,” “an” and “the” include plural references unless the context clearly dictates otherwise.

As used herein, “encapsulated” means enclosed within an outer shell, layer, coating, wall, capsule, matrix, casing, envelope, membrane, barrier, or outer compartment. In some embodiments, an encapsulated material may be provided in the form of a core-shell particle, comprising an inner core, fill, payload, nucleus, reservoir, interior phase, active domain, or inner compartment, and an outer shell, layer, coating, wall, capsule, matrix, casing, envelope, membrane, barrier, or outer compartment. The shell may be continuous or discontinuous, uniform or non-uniform in thickness, and may fully or partially surround the core.

The shell may function to protect the core material, control or modulate release, enhance stability, improve delivery, provide targeting functionality, or combinations thereof. The core-shell particle may be present as a micro-scale or nano-scale particle, including but not limited to microparticles, nanoparticles, colloids, vesicles, or aggregates thereof. In some embodiments, the shell comprises a metallic, polymeric, lipid, inorganic, organic, or hybrid material, and the core comprises one or more cosmetic or pharmaceutical active agents, carriers, excipients, or combinations thereof.

“Encapsulated,” without any modifier, may refer to full and/or partial enclosure. “Full” enclosure refers to complete coverage or enclosure by the shell, layer, coating, wall, or capsule. “Partial” refers to less than full coverage or enclosure, and includes “partial,” “substantial,” and/or “essential” coverage.

As used herein, the term “substantially” or “essentially” refers to a qualitative condition in which a feature or characteristic is present to an entire or nearly entire extent. In some embodiments, “substantially encapsulated” or “essentially encapsulated” refers to near-complete encapsulation of a substance or compound. For example, a substantially encapsulated particle may be encapsulated to at least about 70%, 75%, 80%, 85%, 90%, 95%, 99%, or 100% of its total surface area or volume. In some embodiments, the term “substantially” refers to the presence of at least about 70%, 75%, 80%, 85%, 90%, 95%, 99%, or 100% of the total range or degree of the referenced feature or characteristic.

As used herein, the term “at least partially” refers to a qualitative condition in which a feature or characteristic is present to a partial extent. In some embodiments, “at least partially encapsulated” refers to partial encapsulation of a substance or compound. For example, an at least partially encapsulated particle may be encapsulated to at least about 5%, 10%, 20%, 30%, 40%, 50%, 60%, 70%, 75%, 80%, 85%, 90%, 95%, 99%, or 100% of its total surface area or volume.

As used herein, the terms “therapeutic agent,” “active agent,” “active ingredient,” “active pharmaceutical ingredient,” “cosmetic active,” “bioactive,” “pharmaceutical compound,” “API,” “pharmaceutically active agent,” “pharmaceutical,” “drug,” and similar terms may be used interchangeably to refer to a substance that provides a cosmetic, therapeutic, pharmacological, physiological, or functional benefit to the skin, hair, or body. Such benefits may include, without limitation, improving the appearance, condition, structure, or function of the skin or hair, or modulating biological processes associated therewith.

When any such term is used, or when a particular active agent is identified by name or category, it is understood that the recitation includes the active agent per se, as well as cosmetically acceptable and/or pharmaceutically acceptable, functionally active derivatives or related compounds thereof, including without limitation salts, acceptable salts, N-oxides, prodrugs, active metabolites, isomers, fragments, complexes, solvates (such as hydrates), polymorphs, pseudopolymorphs, esters, and combinations thereof, provided such forms are suitable for cosmetic and/or pharmaceutical use.

As used herein, the terms “inactive ingredient,” “carrier,” “excipient,” “vehicle,” and similar terms refer to a substance or component that does not itself provide a primary cosmetic or therapeutic effect, but is included to facilitate formulation, stability, delivery, bioavailability, manufacturability, aesthetics, or performance of one or more active agents. Such components may function, without limitation, as solvents, diluents, dispersants, emulsifiers, stabilizers, preservatives, humectants, penetration enhancers, encapsulating agents, buffers, or combinations thereof.

When any such term is used, it is understood that the recited inactive ingredient, carrier, or excipient may be cosmetically acceptable and/or pharmaceutically acceptable, and suitable for topical, transdermal, or other routes of administration contemplated herein. The terms further include the component per se as well as acceptable derivatives or related forms thereof, including without limitation salts, solvates (such as hydrates), complexes, polymorphs, pseudopolymorphs, and combinations thereof, provided such forms are compatible with cosmetic and/or pharmaceutical formulations.

As used herein, the term “complex” or “complexed” refers to a state in which two or more substances, compounds, molecules, ions, or materials are associated with one another through one or more chemical, physical, electrostatic, coordination, hydrophobic, hydrogen-bonding, affinity-based, or other non-covalent interactions, and, in some embodiments, covalent interactions. A complex may be reversible or irreversible, transient or stable, and may exist in equilibrium with uncomplexed components. As used herein, the term “complexed” refers to a state in which two or more substances, compounds, molecules, ions, or materials are associated with one another through one or more chemical, physical, electrostatic, coordination, hydrophobic, hydrogen-bonding, affinity-based, or other non-covalent interactions, and, in some embodiments, covalent interactions. A complex may be reversible or irreversible, transient or stable, and may exist in equilibrium with uncomplexed components.

As used herein, the term “bound” refers to an association or attachment between two or more substances, compounds, molecules, ions, or materials such that they are held together by one or more covalent or non-covalent interactions, including without limitation ionic interactions, coordination bonds, hydrogen bonds, van der Waals forces, hydrophobic interactions, affinity-based interactions, receptor-ligand interactions, or combinations thereof.

Binding may be direct or indirect, specific or non-specific, reversible or irreversible, and temporary or sustained. Unless expressly stated otherwise, the term “bound” does not require permanent attachment or covalent bonding.

As used herein, the term “time-release” refers to a controlled, regulated, delayed, sustained, extended, or otherwise modified release of one or more substances over a period of time following application or administration, as compared to an immediate or instantaneous release of the same active ingredient or payload. A time-release system may be configured to release a substance continuously, intermittently, progressively, or in response to one or more conditions, and may provide release over minutes, hours, days, or longer durations. Unless expressly stated otherwise, the term “time-release” does not require a specific release profile, duration, or rate, and encompasses sustained-release, extended-release, delayed-release, controlled-release, and modified-release systems.

As used herein, the term “particle size” refers to a characteristic dimension of a particle, typically expressed as a diameter or equivalent spherical diameter, and may be determined using any suitable particle size measurement technique known in the art. Particle size may be reported as an average value, a median value, or a value derived from a particle size distribution, and may be measured by techniques including, without limitation, dynamic light scattering (DLS), laser diffraction, electron microscopy, or other suitable methods.

As used herein, “D50” refers to the median particle size of a particle size distribution, meaning the particle diameter at which 50 percent of the particles in the distribution, by number or by volume, have a particle size smaller than or equal to the stated value and 50 percent have a particle size larger than or equal to the stated value.

As used herein, the term “nanoparticle” refers to a particle having at least one dimension in the nanometer size range. In some embodiments, a nanoparticle has an average particle size or characteristic dimension of less than about 1000 nm, less than about 500 nm, less than about 200 nm, or less than about 100 nm.

As used herein, “wt %” means “weight percent” or “percentage by weight.” Unless otherwise specified, the based for a wt % disclosed herein is the total weight of the disclosed skincare composition.

### DETAILED DESCRIPTION

Aspects of the present disclosure provide skincare compositions capable of enhancing and promoting skin repair mechanisms during sleep. The disclosed skincare compositions contain a dual-action delivery system including time-release and targeted delivery vehicles. The disclosed compositions may also include a dual peptide system including a signal peptide and a carrier peptide. According to some aspects the dual peptide system may be encapsulated, complexed, bound, or otherwise associated with the delivery system. In some aspects, the disclosed compositions may additionally include an oat lipid composition, a niacinamide, and an antioxidant.

### Dual-Action Delivery System

The dual-action delivery system of the disclosed composition contains a time-release peptide delivery vehicle and a targeted peptide delivery vehicle. In some aspects, the time-release peptide delivery vehicle is a gold-encapsulation vehicle in which the peptide is encapsulated in gold. Thus, in some instances, the time-release delivery vehicle comprises or consists of a core-shell particle, in which the shell comprises or consists of gold and the shell comprises or consists of an active ingredient. In some preferred embodiments, the core comprises or consists of a peptide, such as a peptide of the dual peptide system disclosed herein.

In some embodiments, the gold shell is formed from a gold source included in the compositions, such as colloidal gold, gold nanoparticles, gold-containing matrices, or other sources known in the art. Gold particles, including gold nanoparticles present in colloidal gold, possess surface chemical properties that promote interaction with peptides. In particular, gold exhibits a natural affinity for functional groups commonly present in peptides, including sulfur-containing groups, nitrogen-containing groups, and charged or polar moieties. As a result, peptides may associate with gold surfaces through one or more mechanisms including, without limitation, coordination interactions, electrostatic attraction, affinity-based adsorption, hydrophobic interactions, or combinations thereof. These interactions may occur spontaneously under mild condition to result in the described core-shell particle, in which the peptide and gold are complexed or bound together.

According to some preferred embodiments, the gold source comprises or consists of colloidal gold. Colloidal gold may refer to a dispersion of gold particles in a liquid medium, typically water, in which the gold particles are present in the form of discrete micro-scale or nano-scale particles suspended throughout the medium. In some embodiments, colloidal gold comprises metallic gold particles stabilized against aggregation by surface charge, adsorbed ions, or one or more stabilizing agents.

The gold particles in colloidal gold may be present in a range of particle sizes. In some embodiments, the gold particles have an average particle diameter in the nanometer range, such as from about 1 nm to about 200 nm, including from about 2 nm to about 100 nm, from about 5 nm to about 50 nm, or from about 10 nm to about 30 nm, or any combination therein, e.g., 100 nm or less, 50 nm or less, or 30 nm or less. In some embodiments, the colloidal gold comprises nanoparticles having a relatively narrow particle size distribution, while in other embodiments a broader distribution may be present. Colloidal gold particles may be generally spherical, although other particle morphologies, such as quasi-spherical, irregular, or anisotropic shapes, may also be present.

In some embodiments, the colloidal gold comprises gold particles having a D50 particle size of no greater than about 100 nm, including no greater than about 95 nm, 90 nm, 85 nm, 80 nm, 75 nm, 70 nm, 65 nm, 60 nm, 55 nm, 50 nm, 45 nm, 40 nm, 35 nm, 30 nm, 28 nm, 26 nm, 24 nm, 22 nm, 20 nm, 18 nm, 16 nm, 14 nm, 12 nm, 10 nm, or 8 nm. In some embodiments, the colloidal gold comprises gold particles having a D50 particle size of at least about 8 nm, including at least about 10 nm, 12 nm, 14 nm, 16 nm, 18 nm, 20 nm, 22 nm, 24 nm, 26 nm, 28 nm, 30 nm, 35 nm, 40 nm, 45 nm, 50 nm, 55 nm, 60 nm, 65 nm, 70 nm, 75 nm, 80 nm, 85 nm, 90 nm, 95 nm, or 100 nm.

In some embodiments, colloidal gold is supplied as an aqueous dispersion comprising gold particles at a low concentration, optionally in combination with trace amounts of stabilizing agents or buffering components. The colloidal gold may be substantially free of additional metals or impurities and may be suitable for cosmetic or pharmaceutical use.

In some embodiments, the gold source (e.g., colloidal gold) is included in the disclosed skincare composition in an amount of 0.000005 wt % to 0.00003 wt %, preferably 0.000008 wt % to 0.000025 wt %, more preferably 0.00001 wt % to 0.00002 wt %, still more preferably 0.000012 wt % to 0.000019 wt %, even more preferably 0.000013 wt % to 0.000017 wt %, yet more preferably 0.000014 wt % to 0.000016 wt %, or any combination therein, based on a total weight of the disclosed skincare composition. In some embodiments, colloidal gold is present in the disclosed composition in an amount of at least about 0.00001 wt %, including at least about 0.0000105 wt %, at least about 0.000011 wt %, at least about 0.0000115 wt %, at least about 0.000012 wt %, at least about 0.0000125 wt %, at least about 0.000013 wt %, at least about 0.0000135 wt %, at least about 0.000014 wt %, or at least about 0.000015 wt %, based on a total weight of the disclosed composition. In some embodiments, the gold source (e.g., colloidal gold) is present in the disclosed composition in an amount of no greater than about 0.00003 wt %, including no greater than about 0.000028 wt %, no greater than about 0.000026 wt %, no greater than about 0.000024 wt %, no greater than about 0.000022 wt %, no greater than about 0.000020 wt %, no greater than about 0.000018 wt %, no greater than about 0.000017 wt %, no greater than about 0.000016 wt %, or no greater than about 0.000015 wt %, based on a total weight of the disclosed composition. In a preferred embodiment, the gold source (e.g., colloidal gold) is included in an amount of about 0.000015 wt %.

If the amount of the gold source included in the composition is too low, the gold source may be insufficient to effectively associate with, encapsulate, or stabilize the peptide core. As a result, the peptide may be more susceptible to premature degradation, reduced stability, or uncontrolled release, which can diminish the intended time-release profile and overall performance of the composition. Insufficient gold source may also result in incomplete formation of core-shell particles, leading to variability in delivery and reduced reproducibility of the cosmetic or pharmaceutical effect.

Conversely, if the amount of the gold source is too high, excess gold may lead to particle aggregation, altered particle size distribution, or non-uniform shell formation, which can negatively affect stability, delivery, and release characteristics. Elevated levels of gold source may also interfere with the bioavailability or activity of the peptide, reduce formulation aesthetics, increase manufacturing costs, and result in unnecessary waste of an expensive material. Accordingly, controlling the amount of the gold source within the disclosed ranges may be important for achieving a balance between effective encapsulation, controlled release, stability, performance, and cost efficiency of the composition.

The gold shell may fully or partially encapsulate the peptide core, thereby at least partially isolating the peptide from the surrounding environment. Such encapsulation may protect the peptide from enzymatic degradation, oxidation, or premature interaction with other formulation components and may also allow for a sustained release of peptides over time, ensuring that they remain active in the skin for an extended period.

In some aspects the targeted delivery vehicle may include a peptide, or a moiety, complex, or ligand, e.g., of a peptide or other payload, which is capable of binding to at least one cell responsible for collagen and/or elastin production. For instance, the targeted delivery vehicle may include the lipid moiety to the lipid-modified peptides disclosed herein. In another instance, the targeted delivery vehicle may include the peptide of the metal-binding peptides disclosed herein.

In a preferred embodiment, the targeted delivery vehicle may be included in a stoichiometric amount based on the peptide (or other payload) provided for targeted delivery (e.g. the carrier peptide, disclosed below) which it is bound to, complexed with, or otherwise associated with. In some embodiments, the molar ratio of the delivery vehicle, e.g., targeted delivery vehicle, to the peptide is from about 0.5:1 to about 2:1, including from about 0.6:1 to about 1.8:1, from about 0.7:1 to about 1.6:1, from about 0.8:1 to about 1.4:1, from about 0.85:1 to about 1.3:1, from about 0.9:1 to about 1.2:1, from about 0.95:1 to about 1.1:1, from about 0.97:1 to about 1.05:1, from about 0.98:1 to about 1.02:1, or any combination therein. In some embodiments, the molar ratio of the delivery vehicle to the peptide is at least about 0.5:1, including at least about 0.6:1, at least about 0.7:1, at least about 0.8:1, at least about 0.85:1, at least about 0.9:1, at least about 0.95:1, at least about 0.97:1, at least about 0.98:1, or at least about 1:1. In some embodiments, the molar ratio of the delivery vehicle to the peptide is no greater than about 2:1, including no greater than about 1.8:1, no greater than about 1.6:1, no greater than about 1.4:1, no greater than about 1.3:1, no greater than about 1.2:1, no greater than about 1.1:1, no greater than about 1.05:1, no greater than about 1.02:1, or no greater than about 1:1.

Maintaining the molar ratio of the targeted delivery vehicle to the peptide within the disclosed ranges may be important to achieve effective association with the peptide. If the ratio is too low, insufficient delivery vehicle may be available to adequately associate with the peptide, resulting in reduced stability or a lack of appropriately targeted delivery. If the ratio is too high, excess delivery vehicle may lead to costly inefficient use of materials. Accordingly, controlling the molar ratio within the disclosed ranges may promote targeted delivery, reproducible performance, and efficient use of formulation components.

This targeted approach may ensure that essential peptides are delivered to specific skin cells (e.g. fibroblasts), promoting the production of Collagen I and III. Accordingly, this aspect of the dual-action delivery system may avoid random dispersion of active ingredients through the skin, maximizing the efficacy of the peptides and allowing for sustained collagen stimulation for up to six days post-application.

### Dual Peptide System

The disclosed skincare compositions may also include a dual peptide system including at least two classes of peptide. In some aspects, the dual peptide system includes a signal pepti de and a carrier peptide.

In some embodiments, the signal peptide includes a pentapeptide such as at least one of palmitoyl pentapeptide-3, pentapeptide-18, palmitoyl pentapeptide-12, or acetyl pentapeptide-1. According to some embodiments, non-pentapeptide signal peptides such as at least one of palmitoyl tetrapeptide-7, palmitoyl tripeptide-1, or hexapeptide-9, may be included additionally or alternatively to the pentapeptide. The signal peptide may be capable of stimulating the skin's natural production of Collagen IV, which plays a critical role in reinforcing skin structure and elasticity. Without wishing to be bound by theory, in some instances, the signal peptide peptide may mimic fragments of extracellular matrix (ECM) proteins that are naturally generated during collagen breakdown. Thus, introduction of the signal peptide to the skin may promote skin smoothness, plumpness, and reduce the appearance of fine lines and wrinkles by stimulating the skin's own regenerative processes. In a preferred embodiment, the signal peptide may include palmitoyl pentapeptide-4.

In some embodiments, the signal peptide is included in the disclosed skincare composition in an amount of from about 0.00001 wt % to about 0.00005 wt %, including from about 0.000015 wt % to about 0.000045 wt %, from about 0.000018 wt % to about 0.000042 wt %, from about 0.00002 wt % to about 0.00004 wt %, from about 0.000022 wt % to about 0.000038 wt %, from about 0.000024 wt % to about 0.000036 wt %, from about 0.000026 wt % to about 0.000034 wt %, from about 0.000028 wt % to about 0.000032 wt %, from about 0.000029 wt % to about 0.000031 wt %, about 0.000030 wt %, or any combination therein, based on a total weight of the disclosed skincare composition. In some embodiments, the signal peptide is included in the disclosed skincare composition in an amount of at least about 0.00001 wt %, including at least about 0.000015 wt %, at least about 0.000018 wt %, at least about 0.00002 wt %, at least about 0.000022 wt %, at least about 0.000024 wt %, at least about 0.000026 wt %, at least about 0.000028 wt %, at least about 0.000029 wt %, or at least about 0.000030 wt %, based on a total weight of the disclosed skincare composition. In some embodiments, the signal peptide is included in the disclosed skincare composition in an amount of no greater than about 0.00005 wt %, including no greater than about 0.000045 wt %, no greater than about 0.000042 wt %, no greater than about 0.00004 wt %, no greater than about 0.000038 wt %, no greater than about 0.000036 wt %, no greater than about 0.000034 wt %, no greater than about 0.000032 wt %, no greater than about 0.000031 wt %, or no greater than about 0.000030 wt %, based on a total weight of the disclosed skincare composition. In a preferred embodiment, the signal peptide is included in an amount of 0.000030 wt %.

The carrier peptide may be configured to transport essential trace elements necessary for skin health to the cells responsible for collage and elastin synthesis. In some embodiments, the carrier peptide is included in the disclosed skincare composition in an amount of from about 0.00005 wt % to about 0.0002 wt %, including from about 0.00007 wt % to about 0.00015 wt %, from about 0.00008 wt % to about 0.00014 wt %, from about 0.00009 wt % to about 0.00013 wt %, from about 0.000095 wt % to about 0.00012 wt %, from about 0.000097 wt % to about 0.00011 wt %, from about 0.000098 wt % to about 0.000105 wt %, from about 0.000099 wt % to about 0.000102 wt %, about 0.0001 wt %, or any combination therein, based on a total weight of the disclosed skincare composition.

In some embodiments, the carrier peptide is included in the disclosed skincare composition in an amount of at least about 0.00005 wt %, including at least about 0.00006 wt %, at least about 0.00007 wt %, at least about 0.00008 wt %, at least about 0.00009 wt %, at least about 0.000095 wt %, at least about 0.000097 wt %, at least about 0.000098 wt %, at least about 0.000099 wt %, or at least about 0.0001 wt %, based on a total weight of the disclosed skincare composition. In some embodiments, the carrier peptide is included in the disclosed skincare composition in an amount of no greater than about 0.0002 wt %, including no greater than about 0.00018 wt %, no greater than about 0.00016 wt %, no greater than about 0.00015 wt %, no greater than about 0.00014 wt %, no greater than about 0.00013 wt %, no greater than about 0.00012 wt %, no greater than about 0.00011 wt %, no greater than about 0.000105 wt %, or no greater than about 0.0001 wt %, based on a total weight of the disclosed skincare composition.

According to some embodiments, the carrier peptide may at least one of a metal-binding or lipid-modified peptide and in some preferred embodiments, the carrier peptide includes both a metal-binding and a lipid-modified peptide. In some embodiments, the carrier peptide may include a heptapeptide, e.g., at least one of, or both of, the metal-binding and/or the lipid-modified peptide may include or be a heptapeptide.

Metal-binding peptides are peptides configured to associate with one or more metal ions, such as copper, through coordination interactions with amino acid side chains. Such peptides may facilitate delivery of metal ions that function as cofactors in enzymatic processes associated with skin structure, repair, and antioxidant activity, while stabilizing the metal ions and reducing undesirable interactions within the formulation. According to some embodiments, the metal-binding peptide may comprise a copper-binding peptide such as at least one of copper tripeptide-1, copper heptapeptide-14, copper lysinate/prolinate complexes, or the like.

Lipid-modified peptides are peptides conjugated to one or more lipid moieties, such as fatty acids, which increase peptide lipophilicity and improve compatibility with the lipid-rich environment of the skin. Such modification may enhance skin penetration, retention, and residence time, and may support controlled or sustained delivery of the peptide. According to some embodiments, the lipid-modified peptide may include one or more of heptapeptide-15 palmitate, palmitoyl tripeptide-1, palmitoyl tetrapeptide-7, or palmitoyl hexapeptide-12.

When used together, metal-binding peptides and lipid-modified peptides may provide a synergistic effect that enhances delivery, stability, and functional performance beyond that achievable by either peptide type alone. In combination, the lipid-modified peptide may facilitate improved residence time and distribution of the metal-binding peptide and its associated metal ion within the skin, while the metal-binding peptide may support sustained biological activity at the targeted site. This cooperative interaction may result in more efficient delivery of essential cofactors, prolonged activity, and enhanced support of skin structure and appearance in cosmetic compositions.

In some aspects, the carrier peptide may include at least one of copper heptapeptide-14 pantothenate or heptapeptide-15 palmitate. In a preferred embodiment, the carrier peptide includes copper heptapeptide-14 pantothenate and heptapeptide-15 palmitate, as metal-binding and lipid-modified peptides. When the carrier peptide includes both a metal-binding peptide and a lipid-modified peptide, in some embodiments the metal-binding peptide and the lipid-modified peptide may be used in a weight ratio of from about 10:90 to about 90:10, including from about 20:80 to about 80:20, from about 30:70 to about 70:30, from about 35:65 to about 65:35, from about 40:60 to about 60:40, from about 45:55 to about 55:45, from about 48:52 to about 52:48, about 50:50, or any combination therein.

In some embodiments, the metal-binding peptide is present in an amount of at least about 10 wt % relative to the total weight of the metal-binding peptide and lipid-modified peptide, including at least about 20 wt %, at least about 30 wt %, at least about 35 wt %, at least about 40 wt %, at least about 45 wt %, at least about 48 wt %, or at least about 50 wt %. In some embodiments, the metal-binding peptide is present in an amount of no greater than about 90 wt % relative to the total weight of the metal-binding peptide and lipid-modified peptide, including no greater than about 80 wt %, no greater than about 70 wt %, no greater than about 65 wt %, no greater than about 60 wt %, no greater than about 55 wt %, no greater than about 52 wt %, or no greater than about 50 wt %.

Maintaining the weight ratio of the metal-binding peptide to the lipid-modified peptide within the disclosed ranges may be important to preserve the intended cooperative function of the carrier peptide system. When the ratio is too low, the proportion of metal-binding peptide may be insufficient relative to the lipid-modified peptide, which can result in inadequate delivery or availability of the associated metal component and reduced support for metal-dependent processes, thereby diminishing the effectiveness of the carrier peptide system. In such cases, the system may rely disproportionately on the lipid-modified peptide, leading to reduced functional balance.

Conversely, when the ratio is too high, the proportion of metal-binding peptide may be excessive relative to the lipid-modified peptide, which can limit the penetration, retention, or distribution benefits provided by the lipid-modified peptide. Excess metal-binding peptide may also increase the likelihood of aggregation or undesirable interactions within the formulation. In this scenario, the relative amount of lipid-modified peptide may be insufficient to effectively facilitate delivery and residence within the skin. Accordingly, controlling the weight ratio within the disclosed ranges may ensure that neither peptide predominates to the detriment of the other, thereby supporting balanced delivery, stability, and consistent performance of the skincare composition.

The combination of signal and carrier peptides may provide a synergistic effect, in which the signal peptides may stimulate collagen production and the carrier peptides ensure that the correct nutrients are delivered to specific targeted cells or cell types in order to maximize this effect. In order to achieve this synergistic effect, it is important that the signal peptide and the carrier peptide be included in appropriate amounts. When the amount of signal peptide relative to carrier peptide is too low, this can result in reduced stimulation of skin signaling pathways and diminished activation of processes associated with skin structure and renewal, despite the presence of an effective delivery system. Conversely, when the same relative amount is too high, the amount of signal peptide may be excessive relative to the carrier peptide, which can limit effective transport, localization, or sustained availability of the signal peptide within the skin. In such cases, the signal peptide may be delivered inefficiently, released prematurely, or degraded before achieving its intended effect.

Accordingly, in some embodiments, a weight ratio of the signal peptide to the carrier peptide is from about 0.05:1 to about 1:1, including from about 0.067:1 to about 0.8:1, from about 0.1:1 to about 0.6:1, from about 0.15:1 to about 0.5:1, from about 0.2:1 to about 0.4:1, from about 0.25:1 to about 0.35:1, from about 0.28:1 to about 0.33:1, from about 0.29:1 to about 0.31:1, from about 0.295:1 to about 0.305:1, about 0.3:1, or any combination therein. In some embodiments, the weight ratio of the signal peptide to the carrier peptide is at least about 0.05:1, including at least about 0.1:1, at least about 0.15:1, at least about 0.2:1, at least about 0.25:1, at least about 0.28:1, at least about 0.29:1, at least about 0.295:1, at least about 0.3:1. In some embodiments, the weight ratio of the signal peptide to the carrier peptide is no greater than about 1:1, including no greater than about 0.8:1, no greater than about 0.6:1, no greater than about 0.5:1, no greater than about 0.4:1, no greater than about 0.35:1, no greater than about 0.33:1, no greater than about 0.31:1, or no greater than about 0.3:1.

In some embodiments, the dual peptide system may be combined with the dual-action delivery system such that the signal peptides are paired with the gold encapsulation vehicle and the carrier peptides utilize the targeted delivery vehicle. The gold encapsulated signal peptides may stimulate the production of Collagen IV, which plays a key role in the skin's structural integrity, resulting in a smoother, more resilient complexion and a reduction in the appearance of fine lines and wrinkles. At the same time, pairing of the carrier peptides with the targeted delivery system may ensure delivery of nutrients to specific targets.

### Niacinamide

In some embodiments, niacinamide is included in the disclosed skincare composition in an amount of from about 1.0 wt % to about 4.0 wt %, including from about 1.5 wt % to about 3.5 wt %, from about 1.8 wt % to about 3.2 wt %, from about 2.0 wt % to about 3.0 wt %, from about 2.1 wt % to about 2.9 wt %, from about 2.2 wt % to about 2.8 wt %, from about 2.3 wt % to about 2.7 wt %, from about 2.4 wt % to about 2.6 wt %, from about 2.45 wt % to about 2.55 wt %, about 2.50 wt %, or any combination therein, based on a total weight of the disclosed skincare composition.

In some embodiments, niacinamide is included in the disclosed skincare composition in an amount of at least about 1.0 wt %, including at least about 1.2 wt %, at least about 1.5 wt %, at least about 1.8 wt %, at least about 2.0 wt %, at least about 2.2 wt %, at least about 2.3 wt %, at least about 2.4 wt %, at least about 2.45 wt %, or at least about 2.50 wt %, based on a total weight of the disclosed skincare composition. In some embodiments, niacinamide is included in the disclosed skincare composition in an amount of no greater than about 4.0 wt %, including no greater than about 3.8 wt %, no greater than about 3.5 wt %, no greater than about 3.2 wt %, no greater than about 3.0 wt %, no greater than about 2.8 wt %, no greater than about 2.7 wt %, no greater than about 2.6 wt %, no greater than about 2.55 wt %, or no greater than about 2.50 wt %, based on a total weight of the disclosed skincare composition.

When niacinamide is included within the disclosed ranges, it is present in the disclosed skincare composition at an amount which may allow for compatibility with sensitive skin as well as synergistic interactions with other active ingredients. In the disclosed amounts, niacinamide may be useful to improve overall skin texture, reduce hyperpigmentation, and strengthen the skin's barrier without causing irritation. Barrier repair is a key component of overnight skin recovery, which may be compromised in sleep-deprived skin.

### Lipids

In some aspects the disclosed compositions may include an oat lipid composition. In some embodiments, the oat lipid composition may include an oat extract, an oat oil, and/or an oat flour. In a preferred embodiment, the oat lipid composition includes an oat extract, an oat oil, and/or an oat flour. Preferably, the oat lipid composition includes Avena sativa (oat) kernel extract, Avena sativa (oat) kernel oil, and Avena sativa (oat) kernel flour.

In some embodiments, the oat lipid composition is included in the disclosed skincare composition in an amount of from about 1.0 wt % to about 4.0 wt %, including from about 1.5 wt % to about 3.5 wt %, from about 1.8 wt % to about 3.2 wt %, from about 2.0 wt % to about 3.0 wt %, from about 2.1 wt % to about 2.9 wt %, from about 2.2 wt % to about 2.8 wt %, from about 2.3 wt % to about 2.7 wt %, from about 2.4 wt % to about 2.6 wt %, from about 2.45 wt % to about 2.55 wt %, about 2.50 wt %, or any combination therein, based on a total weight of the disclosed skincare composition.

In some embodiments, the oat lipid composition is included in the disclosed skincare composition in an amount of at least about 1.0 wt %, including at least about 1.2 wt %, at least about 1.5 wt %, at least about 1.8 wt %, at least about 2.0 wt %, at least about 2.2 wt %, at least about 2.3 wt %, at least about 2.4 wt %, at least about 2.45 wt %, or at least about 2.50 wt %, based on a total weight of the disclosed skincare composition. In some embodiments, the oat lipid composition is included in the disclosed skincare composition in an amount of no greater than about 4.0 wt %, including no greater than about 3.8 wt %, no greater than about 3.5 wt %, no greater than about 3.2 wt %, no greater than about 3.0 wt %, no greater than about 2.8 wt %, no greater than about 2.7 wt %, no greater than about 2.6 wt %, no greater than about 2.55 wt %, or no greater than about 2.50 wt %, based on a total weight of the disclosed skincare composition.

When the oat lipid composition is included within the disclosed ranges, it may serve to soothe and nourish the skin, enhancing the effect of the other actives by making the skin more receptive to them. Further, the oat lipid composition may enhance the skin's ability to retain moisture and protect itself from environmental stressors.

In some embodiments, evening primrose oil is included in the disclosed skincare composition in an amount of from about 0.5 wt % to about 3.0 wt %, including from about 0.6 wt % to about 2.5 wt %, from about 0.65 wt % to about 2.2 wt %, from about 0.7 wt % to about 2.0 wt %, from about 0.75 wt % to about 1.8 wt %, from about 0.8 wt % to about 1.6 wt %, from about 0.85 wt % to about 1.4 wt %, from about 0.9 wt % to about 1.2 wt %, from about 0.95 wt % to about 1.05 wt %, about 1.0 wt %, or any combination therein, based on a total weight of the disclosed skincare composition.

In some embodiments, evening primrose oil is included in the disclosed skincare composition in an amount of at least about 0.5 wt %, including at least about 0.6 wt %, at least about 0.7 wt %, at least about 0.8 wt %, at least about 0.85 wt %, at least about 0.9 wt %, at least about 0.95 wt %, or at least about 1.0 wt %, based on a total weight of the disclosed skincare composition. In some embodiments, evening primrose oil is included in the disclosed skincare composition in an amount of no greater than about 3.0 wt %, including no greater than about 2.8 wt %, no greater than about 2.5 wt %, no greater than about 2.2 wt %, no greater than about 2.0 wt %, no greater than about 1.8 wt %, no greater than about 1.6 wt %, no greater than about 1.4 wt %, no greater than about 1.2 wt %, no greater than about 1.05 wt %, or no greater than about 1.0 wt %, based on a total weight of the disclosed skincare composition. Evening primrose oil is rich in gamma-linolenic acid (GLA) and, when included within the disclosed ranges, it may serve to nourish and repair the skin barrier.

In some aspects the disclosed compositions may include jojoba seed oil. In some embodiments, the jojoba seed oil is included in the disclosed skincare composition in an amount of from about 0.5 wt % to about 3.0 wt %, including from about 0.6 wt % to about 2.5 wt %, from about 0.65 wt % to about 2.2 wt %, from about 0.7 wt % to about 2.0 wt %, from about 0.75 wt % to about 1.8 wt %, from about 0.8 wt % to about 1.6 wt %, from about 0.85 wt % to about 1.4 wt %, from about 0.9 wt % to about 1.2 wt %, from about 0.95 wt % to about 1.05 wt %, about 1.0 wt %, or any combination therein, based on a total weight of the disclosed skincare composition.

In some embodiments, jojoba seed oil is included in the disclosed skincare composition in an amount of at least about 0.5 wt %, including at least about 0.6 wt %, at least about 0.7 wt %, at least about 0.8 wt %, at least about 0.85 wt %, at least about 0.9 wt %, at least about 0.95 wt %, or at least about 1.0 wt %, based on a total weight of the disclosed skincare composition. In some embodiments, jojoba seed oil is included in the disclosed skincare composition in an amount of no greater than about 3.0 wt %, including no greater than about 2.8 wt %, no greater than about 2.5 wt %, no greater than about 2.2 wt %, no greater than about 2.0 wt %, no greater than about 1.8 wt %, no greater than about 1.6 wt %, no greater than about 1.4 wt %, no greater than about 1.2 wt %, no greater than about 1.05 wt %, or no greater than about 1.0 wt %, based on a total weight of the disclosed skincare composition.

In some aspects the disclosed compositions may include safflower oleosomes. In some embodiments, safflower oleosomes are included in the disclosed skincare composition in an amount of from about 0.1 wt % to about 1.50 wt %, including from about 0.2 wt % to about 1.20 wt %, from about 0.25 wt % to about 1.05 wt %, from about 0.3 wt % to about 0.90 wt %, from about 0.35 wt % to about 0.85 wt %, from about 0.4 wt % to about 0.8 wt %, from about 0.45 wt % to about 0.75 wt %, from about 0.5 wt % to about 0.7 wt %, from about 0.55 wt % to about 0.65 wt %, about 0.60 wt %, or any combination therein, based on a total weight of the disclosed skincare composition.

In some embodiments, safflower oleosomes are included in the disclosed skincare composition in an amount of at least about 0.1 wt %, including at least about 0.2 wt %, at least about 0.25 wt %, at least about 0.3 wt %, at least about 0.35 wt %, at least about 0.4 wt %, at least about 0.45 wt %, at least about 0.5 wt %, at least about 0.55 wt %, or at least about 0.60 wt %, based on a total weight of the disclosed skincare composition. In some embodiments, safflower oleosomes are included in the disclosed skincare composition in an amount of no greater than about 1.50 wt %, including no greater than about 1.35 wt %, no greater than about 1.20 wt %, no greater than about 1.05 wt %, no greater than about 0.90 wt %, no greater than about 0.85 wt %, no greater than about 0.80 wt %, no greater than about 0.75 wt %, no greater than about 0.70 wt %, no greater than about 0.65 wt %, or no greater th

### Antioxidants

In some aspects, the disclosed compositions may include an antioxidant. Nonlimiting examples of antioxidants include tocopherols and their derivatives, ascorbic acid and its derivatives, carotenoids, flavonoids, anthocyanins, cinnamic acid and its derivatives, and the like.

For example, tocopherols and their derivatives may include, e.g., d-α-tocopherol, d-β-tocopherol, d-γ-tocopherol, or d-delta-tocopherol; tocotrienols (e.g., d-α-tocotrienol, d-β-tocotrienol, d-γ.-tocotrienol, d-delta-tocotrienol); and vitamin E (α-tocopherol acetate). These compounds may be isolated from natural sources, prepared by synthetic means, or combinations thereof. Useful tocotrienols are natural products isolated, for example, from wheat germ oil, grain, or palm oil. As used herein, the term “tocotrienols” includes tocotrienol-rich-fractions obtained from these natural products as well as the pure compounds.

In addition to ascorbic acid (Vitamin C), ascorbic acid derivatives may be used, including sodium ascorbate, and the fat soluble esters tetrahexyldecyl ascorbate and ascorbyl palmitate, magnesium ascorbyl phosphate, ascorbyl-glucoside, glucosamine ascorbate, ascorbyl acetate, etc. Additionally, extracts from plants containing a high amount of vitamin C such as camu berry (Myrciaria dubia), acerola, Emblica officinalis, and bioflavonoids from rose hip and citrus may be used including water soluble bioflavonoids such as hesperidin methyl chalcone may also be used.

In addition, carotenoids, particularly the xanthophyll type, may also be included. The xanthopyll type carotenoids include molecules, such as lutein, canthaxantin, cryptoxanthin, zeaxanthin and astaxanthin. Xanthophylls protect compounds, such as vitamin A, vitamin E, and other carotenoids. Carotenoids may also include: beta-carotene, alpha-carotene, gamma-carotene, beta-cryptoxanthin, lycopene, lutein, and idebenone.

Flavonoids may also be used as antioxidants. The flavonoid may be a flavanone, a flavanol, and/or a flavone.

The antioxidant may also include an anthocyanin. Anthocyanins and their derivatives are antioxidants. Anthocyanins encompasses a class of flavonoid compounds that are naturally occurring, water-soluble compounds, responsible for the red, purple, and blue colors of many fruits, vegetables, cereal grains, and flowers. Additionally, anthocyanins are collagenase inhibitors. The inhibition of collagenase helps in the prevention and reduction of wrinkles, increase in skin elasticity, etc., which are caused by a reduction in skin collagen. The anthocyanins may be obtained from any portion of various plant sources, such as the fruit, flower, stem, leaves, root, bark, or seeds. In some instances, antioxidants may include one or more betacyanin. Betacyanins, like anthocyanins, may be obtained from natural sources and are antioxidants.

The antioxidant may also include cinnamic acid and/or its derivatives, including cinnamic acid, caffeic acid, ferulic acid, trans-ferulic acid, 5-hydroxyferulic acid, sinapic acid, coumaryl alcohol, coniferyl alcohol, sinapyl alcohol, eugenol, chavicol, safrole, p-coumaric acid, and sinapinic acid.

The antioxidant may also include a curcuminoid. Curcuminoids may include: curcumin, desmethoxycurcumin, bis-desmethoxycurcumin, tetrahydrocurcumin, and tetrahydrocurcuminoids.

The antioxidant may further include one or more of allantoin, kojic acid, salicylic acid, or glutathione.

### Solvents

In a preferred embodiment, the disclosed skincare composition is an aqueous composition, including water as a solvent. In some embodiments, the solvent is included in the disclosed skincare composition in an amount of from about 40 wt % to about 90 wt %, including from about 50 wt % to about 80 wt %, from about 55 wt % to about 78 wt %, from about 60 wt % to about 75 wt %, from about 62 wt % to about 74 wt %, from about 64 wt % to about 73 wt %, from about 66 wt % to about 72 wt %, from about 68 wt % to about 71 wt %, from about 69 wt % to about 70.5 wt %, about 70 wt %, or any combination therein, based on a total weight of the disclosed skincare composition.

In some embodiments, the solvent is included in the disclosed skincare composition in an amount of at least about 40 wt %, including at least about 45 wt %, at least about 50 wt %, at least about 55 wt %, at least about 60 wt %, at least about 62 wt %, at least about 64 wt %, at least about 66 wt %, at least about 68 wt %, at least about 69 wt %, or at least about 70 wt %, based on a total weight of the disclosed skincare composition. In some embodiments, the solvent is included in the disclosed skincare composition in an amount of no greater than about 90 wt %, including no greater than about 85 wt %, no greater than about 80 wt %, no greater than about 78 wt %, no greater than about 75 wt %, no greater than about 74 wt %, no greater than about 73 wt %, no greater than about 72 wt %, no greater than about 71 wt %, or no greater than about 70 wt %, based on a total weight of the disclosed skincare composition.

In some embodiments, one or more additional solvents may be included in the disclosed skincare composition in addition to water. Such additional solvents may include glycols, diols, polyols, or combinations thereof, including, without limitation, propanediol, butylene glycol, pentylene glycol, hexylene glycol, or combinations thereof. The additional solvents are selected to be safe and suitable for cosmetic and/or pharmaceutical use. These additional solvents may function to improve solubility of one or more components, enhance formulation stability, support uniform dispersion of active ingredients, modify sensory properties, or assist in processing and manufacturing of the composition.

### Humectants

In some embodiments, a humectant may also be included in the disclosed skincare composition. A humectant is a substance that attracts and retains moisture from the environment and draws it into the skin or other surfaces. Examples include glycerin, hyaluronic acid, sorbitol, urea, aloe vera, and glycols having from 2 to 8 carbon atoms, monosaccharides, disaccharides, and sugar alcohols.

The total amount of humectant in the cosmetic composition will vary but, in some embodiments, the total amount of humectant is included in the disclosed skincare composition in an amount of from about 0.1 wt % to about 15 wt %, including from about 0.1 wt % to about 10 wt %, from about 1 wt % to about 10 wt %, from about 3 wt % to about 10 wt %, from about 5 wt % to about 10 wt %, from about 6 wt % to about 10 wt %, from about 7 wt % to about 10 wt %, from about 8 wt % to about 10 wt %, from about 9 wt % to about 10 wt %, about 10 wt %, or any combination therein, based on a total weight of the disclosed skincare composition.

In some embodiments, the humectant is included in the disclosed skincare composition in an amount of at least about 0.1 wt %, including at least about 0.5 wt %, at least about 1 wt %, at least about 3 wt %, at least about 5 wt %, at least about 6 wt %, at least about 7 wt %, at least about 8 wt %, at least about 9 wt %, or at least about 10 wt %, based on a total weight of the disclosed skincare composition. In some embodiments, the humectant is included in the disclosed skincare composition in an amount of no greater than about 15 wt %, including no greater than about 12 wt %, no greater than about 10 wt %, no greater than about 9 wt %, no greater than about 8 wt %, no greater than about 7 wt %, no greater than about 6 wt %, no greater than about 5 wt %, or no greater than about 3 wt %, based on a total weight of the disclosed skincare composition.

### Additives

In addition to the components described above, the disclosed skincare compositions may further include one or more optional additives to support formulation stability, aesthetics, preservation, and overall performance. For instance, in some embodiments, the composition may include one or more silicones or silicone elastomers, such as dimethicone or crosslinked silicones, which may contribute to spreadability, texture, and sensory properties. In some embodiments, the composition may further include one or more emulsifiers, surfactants, or solubilizing agents, including ethoxylated castor oil derivatives, glyceryl esters, phospholipids, or sugar-based surfactants, to support dispersion of lipid components within the aqueous phase.

In some embodiments, the composition may include one or more thickening agents or rheology modifiers, such as carbomers, xanthan gum, polyvinyl alcohol, or combinations thereof, to adjust viscosity and stabilize emulsified or suspended components. One or more polymeric film-formers may also be included to enhance skin feel or deposition.

In some embodiments, the composition may include one or more preservatives or preservation systems, such as phenoxyethanol, organic acids, glycols, or salt-based preservatives, as well as one or more chelating agents to improve formulation stability. The composition may further include one or more pH-adjusting or buffering agents, such as acids, bases, or salts, to maintain the composition within a desired pH range.

In some embodiments, the composition may optionally include additional skin-benefit additives, such as soothing agents, exfoliating agents, botanical extracts, or combinations thereof, provided such additives are compatible with the active ingredients and delivery systems described herein.

In some embodiments, the disclosed skincare composition may be formulated to exclude one or more ingredients or classes of ingredients. For example, the composition may be free or substantially free of one or more impurities or contaminant classes, including 1,4-dioxane, acrylamides, asbestos, benzene, dioxins, formaldehyde, nitrosamines, residual monomers, residual solvents, free amines, free ethylene oxide, or combinations thereof. In some embodiments, the composition may be free or substantially free of one or more of alkylphenol ethoxylates and their degradation products, glycol ethers, phthalates, pesticides, nitrates, heavy metals, or other non-essential metals. In some embodiments, the composition may further be free or substantially free of one or more of genetically modified organisms (GMOs), gluten, lactose, fragrances, colorants, dyes, parabens, sulfates, dibutyl, toluene, talc, mineral oils, alcohols, silicones, or latex.

### Formulation

In some embodiments, the disclosed skincare composition may be provided in the form of an aqueous cosmetic composition comprising a continuous aqueous phase and one or more dispersed phases. The composition may be provided in the form of a serum, lotion, gel, or similar topical formulation.

In some embodiments, the continuous aqueous phase comprises water and one or more water-soluble or water-dispersible components. Such components may include, without limitation, niacinamide, humectants, salts, buffers, preservatives, antioxidants, peptides present at low concentration, and water-soluble polymers. The aqueous phase may further include dissolved or dispersed metal-binding peptides and water-soluble signal peptides.

In some embodiments, the composition comprises a dispersed lipid phase present as emulsified droplets within the aqueous phase. The lipid phase may include one or more plant-derived oils, lipid-modified peptides, silicones, or combinations thereof. In some embodiments, the lipid phase includes lipid-modified peptides, such as palmitoylated peptides, and one or more lipid components selected from oat-derived lipids, jojoba oil, evening primrose oil, oleosomes, or combinations thereof. The lipid phase may be stabilized by one or more emulsifiers or surfactants.

In some embodiments, the composition further comprises dispersed particulate or colloidal components. Such components may include structured lipid particles, oleosomes, polymeric particles, thickeners, and encapsulated delivery particles. In some embodiments, a time-release delivery system comprises core-shell particles in which one or more signal peptides are at least partially encapsulated within a gold-based shell, forming colloidal or nano-scale particles dispersed within the aqueous phase. In some instances, the core-shell particles may be suspended in the aqueous phase, such that the composition is in the form of a transparent suspension.

Accordingly, the disclosed skincare composition may be characterized as an aqueous dispersion comprising dissolved aqueous-phase components, emulsified lipid-phase components, and suspended or colloidal particulate components, wherein different classes of active agents are distributed among the phases to support stability, delivery, and controlled release.

In some embodiments, the disclosed skincare composition has a viscosity of from about 7,600 cps to about 11,500 cps, including from about 8,000 cps to about 11,000 cps, from about 8,400 cps to about 10,600 cps, from about 8,800 cps to about 10,200 cps, from about 9,000 cps to about 10,000 cps, from about 9,200 cps to about 9,800 cps, from about 9,300 cps to about 9,700 cps, from about 9,400 cps to about 9,600 cps, about 9,500 cps, or any combination therein.

In some embodiments, the disclosed skincare composition has a viscosity of at least about 7,600 cps, including at least about 8,000 cps, at least about 8,400 cps, at least about 8,800 cps, at least about 9,000 cps, at least about 9,200 cps, at least about 9,300 cps, at least about 9,400 cps, or at least about 9,500 cps. In some embodiments, the disclosed skincare composition has a viscosity of no greater than about 11,500 cps, including no greater than about 11,000 cps, no greater than about 10,600 cps, no greater than about 10,200 cps, no greater than about 10,000 cps, no greater than about 9,800 cps, no greater than about 9,700 cps, no greater than about 9,600 cps, or no greater than about 9,500 cps.

The viscosities disclosed herein may be measured using a Brookfield RV viscometer with a #4 spindle at 10 RPM for 1 minute, at a controlled temperature, such as about 20° C. to about 25° C. According to some aspects, the disclosed compositions may demonstrate stability, e.g., in that the measured viscosity may be constant, or substantially constant at the time of manufacturing and at 24 hours after the time of manufacturing. The viscosity may be within the disclosed ranges both at time zero (e.g., the time of manufacturing) and at 24 hours later.

A viscosity within the disclosed ranges may promote stability of emulsified and dispersed components, reduce sedimentation or phase separation, and support uniform distribution of active ingredients, including encapsulated or particulate delivery systems. Additionally, a viscosity within the disclosed ranges may contribute to desirable sensory properties, such as spreadability, skin feel, and ease of application, while supporting retention of the composition on the skin surface for effective delivery of active agents.

In some embodiments, the disclosed skincare composition has a pH of from about 5.0 to about 6.0, including from about 5.1 to about 5.9, from about 5.2 to about 5.8, from about 5.3 to about 5.7, from about 5.4 to about 5.6, about 5.5, or any combination therein.

In some embodiments, the disclosed skincare composition has a pH of at least about 5.0, including at least about 5.1, at least about 5.2, at least about 5.3, at least about 5.4, or at least about 5.5. In some embodiments, the disclosed skincare composition has a pH of no greater than about 6.0, including no greater than about 5.9, no greater than about 5.8, no greater than about 5.7, no greater than about 5.6, or no greater than about 5.5.

Maintaining the pH of the disclosed skincare composition within the specified range may be important for compatibility with the skin's natural acid mantle and for optimal performance of the ingredients included in the composition. A pH within this range may support skin barrier function, reduce the likelihood of irritation, and promote comfort during topical application. Additionally, a pH within the disclosed range may help maintain stability of peptides, niacinamide, botanical extracts, and lipid components, while minimizing degradation or undesirable interactions among formulation components. Fon instance, a mildly acidic pH may help preserve peptide integrity by reducing hydrolysis and maintaining metal-peptide coordination, while also limiting conversion of niacinamide to nicotinic acid. The disclosed pH range may also further reduce degradation of lipid and botanical components and support the performance of emulsifiers, polymers, and stabilizers used to maintain dispersed phases.

### Method of Using the Skincare Composition

In some aspects, a method of using the disclosed skincare composition is provided. The disclosed methods may include use of the disclosed composition to cosmetically treat, address, or improve, a skin conditions or concerns, including signs of aging, fine lines and/or wrinkles, uneven texture, loss of firmness, dryness, or environmental stress-related skin changes.

According to some aspects, the method may comprise applying the skincare composition to a treatment area of the skin. In some embodiments, the skin may be cleansed prior to application using a cleanser suitable for the area of application, although cleansing is not required in all embodiments. In some embodiments, the composition is applied by gently smoothing or spreading the composition onto one or more skin areas, including the face, neck, décolletage, or combinations thereof.

In some embodiments, the composition may be applied in an effective amount. In some instances, the effective amount may be an amount sufficient to coat the desired treatment area. In some cases, effective amount corresponds to approximately one or more pump actuations of a pump, when the composition is stored in a contained in a container having a pump dispenser. In some embodiments, two to three pump actuations may be applied. In some embodiments, a single pump actuation dispenses from about 0.3 mL to about 0.6 mL of the composition, such as from about 0.4 mL to about 0.5 mL, although greater or lesser amounts may be used depending on the dispenser configuration, formulation viscosity, or desired coverage.

In some embodiments, the method may comprise applying the composition once per day. In some embodiments, the composition may be applied more than once per day, twice per day, 3 times per day, 4 times per day, or such as 2 times or more, 3 times or more, 4 times or more, or 5 times or more, and 10 times or less, 9 times or less, 8 times or less, 7 times or less, 6 times or less, 5 times or less, 4 times or less, or any combination therein. In some instances, the composition may be applied once or twice, such as in the morning, in the evening, or both. In some embodiments, the method may be performed daily, every two days, every 3 days, every 4 days, every 5 days, every 6 days, weekly, monthly, otherwise periodically, or according to a personalized skincare routine, and may be continued over a period of days, weeks, or longer.

### EXAMPLES

The following examples are provided to illustrate certain embodiments of the invention and are not intended to limit the scope of the invention in any manner. Those of ordinary skill in the art will recognize that variations in materials, amounts, procedures, and conditions may be made without departing from the spirit and scope of the invention as defined by the claims.

### Example 1—Evaluation of Effect on Fine Lines and Wrinkles

An skincare composition according to the present disclosure, containing a gold-encapsulated palmitoyl pentapeptide-4 (as a signal peptide), a copper heptapeptide-14 pantothenate (as a metal-binding carrier peptide), and heptapeptide-15 palmitate (as a lipid-modified carrier peptide) was evaluated to assess its effect on the appearance of global facial fine lines and wrinkles.

A panel of female subjects between 30 and 65 years of age applied the skincare composition to the face and neck according to standardized use instructions (once in the morning and once in the evening, after cleansing) over a six-week period. Baseline evaluations were conducted prior to use, and follow-up evaluations were conducted after four weeks and six weeks of continued use. Digital facial images were captured at each evaluation using standardized lighting and imaging conditions, and image analysis was performed using computer-assisted software to quantify changes in the appearance of facial fine lines and wrinkles. The measured parameter was a mean area score, with a decrease in the score indicating an improvement in the appearance of fine lines and wrinkles. The results were summarized in Table I below.

TABLE I
Facial Fine Lines/Wrinkles - Image Analysis
Mean Score ± Standard Deviation (S.D.), Mean Change
from Baseline and % of Subjuects and Improvement from Baseline

| | | | | % of Subjects |
|---|---|---|---|---|
| | | | | with |
| | Mean Score ± | | Mean Change | Improvement |
| | S.D. | p-value | from Baseline | from Baseline |
| Baseline | 718.7 ± 145.3 | | | |
| 4 Weeks | 638.5* ± 65.7 | 0.001 | −11.2% | 93% |
| 6 Weeks | 604.1* ± 55.6 | <0.001 | −15.9% | 100% |

*Statistically significant compared with baseline, p ≤ 0.05

As shown in Table I, image analysis demonstrated a statistically significant reduction in the mean fine line and wrinkle score after four weeks of use compared with baseline, corresponding to an average improvement of approximately 11%. After six weeks of use, the mean score showed a further statistically significant reduction compared with baseline, corresponding to an average improvement of approximately 16%. A substantial majority of subjects exhibited improvement at both time points, with nearly all subjects showing improvement after six weeks of use.

### Example 2—Evaluation of Effect on Skin Firmness

The same skincare composition as in Example 1 was further evaluated to assess its effect on skin firmness. A panel of female subjects between 30 and 65 years of age applied the skincare composition to the face and neck according to standardized use instructions (once in the morning and once in the evening, after cleansing) over a six-week period. Baseline measurements were obtained prior to use, and follow-up measurements were obtained after four weeks and six weeks of continued use. Skin firmness was evaluated instrumentally using a suction-based skin measurement device (Cutometer), with firmness assessed using the R0 parameter. A decrease in the R0 value indicates increased skin firmness. The results are summarized in Table II.

TABLE II
Cutometer R0 Measurements
Mean Score ± S.D., Mean Change from Baseline and %
of Subjects with Improvement from Baseline

| | | | | % of Subjects |
|---|---|---|---|---|
| | | | | with |
| | Mean Score ± | | Mean Change | Improvement |
| | S.D. | p-value | from Baseline | from Baseline |
| Baseline | 0.146 ± 0.043 | | | |
| 4 Weeks | 0.130 ± 0.044 | 0.081 | −11.0% | 70% |
| 6 Weeks | 0.084* ± 0.040 | <0.001 | −42.5% | 90% |

*Statistically significant compared with baseline, p ≤ 0.05

As shown in Table II, the mean R0 value decreased after four weeks of use relative to baseline, corresponding to an average improvement in skin firmness of approximately 11%. After six weeks of use, the mean R0 value showed a substantially greater reduction relative to baseline, corresponding to an average improvement in skin firmness of approximately 42%. The improvement observed at six weeks was statistically significant compared with baseline. The charts further show that a majority of subjects exhibited improvement in skin firmness, with approximately 90% of subjects demonstrating improvement after six weeks of use.

### Example 3—Evaluation of Improvement in Skin Hydration

The same skincare composition as in Example 1 was further evaluated to assess its effect on skin hydration. Female subjects applied the skincare composition instructions (once in the morning and once in the evening, after cleansing) to the face and neck according to standardized use instructions over a six-week period. Baseline hydration measurements were obtained prior to use, and follow-up measurements were obtained after four weeks and six weeks of continued use. Skin hydration was measured instrumentally using a capacitance-based skin hydration measurement device (Corneometer). An increase in Corneometer values indicates increased skin moisture. The results are summarized in Table III.

TABLE III
Corneometer Measurements
Mean ± S.D., Mean Change from Baseline and % of
Subjects with Improvement from Baseline

| | | | | % of Subjects |
|---|---|---|---|---|
| | | | | with |
| | Mean Score ± | | Mean Change | Improvement |
| | S.D. | p-value | from Baseline | from Baseline |
| Baseline | 32.1 ± 5.4 | | | |
| 4 Weeks | 38.9* ± 6.8 | <0.001 | 21.2% | 87% |
| 6 Weeks | 38.4* ± 10.3 | 0.006 | 19.6% | 63% |

*Statistically significant compared with baseline, p ≤ 0.05

As shown in Table III, the mean skin hydration value increased significantly after four weeks of use compared with baseline, corresponding to an average increase in hydration of approximately 21%. After six weeks of use, the mean hydration value remained significantly elevated compared with baseline, corresponding to an average increase of approximately 20%. The charts further show that a substantial majority of subjects exhibited increased skin hydration, with approximately 87% of subjects showing improvement after four weeks and approximately 63% showing improvement after six weeks.

Although the invention has been described in detail with reference to certain embodiments, those skilled in the art will appreciate that variations, modifications, and substitutions may be made without departing from the scope of the invention. It is intended that the invention encompass all such variations, modifications, and equivalents as fall within the scope of the appended claims.

What is claimed is:

1. A skincare composition, comprising:
a core-shell particle having a core comprising a signal peptide, the core encapsulated by a shell formed of gold;
a carrier peptide comprising a metal-binding peptide and a lipid-modified peptide;
a niacinamide; and
an oat lipid composition comprising at least one of an oat extract, an oat oil, or an oat flour;
wherein a weight ratio of the signal peptide to the carrier peptide is within a range of 0.05:1 to 1:1, and
wherein a weight ratio of the metal-binding peptide to the lipid-modified peptide is within a range of 40:60 to 60:40.

2. The skincare composition of claim 1, wherein the signal peptide comprises one or more of a palmitoyl pentapeptide-3, a pentapeptide-18, a palmitoyl pentapeptide-12, a acetyl pentapeptide-1, a palmitoyl tetrapeptide-7, a palmitoyl tripeptide-1, or a hexapeptide-9.

3. The skincare composition of claim 2, wherein the signal peptide comprises a palmitoyl pentapeptide-4.

4. The skincare composition of claim 1, wherein the gold is in a form of a nanoparticle having a particle size of no greater than about 200 nm.

5. The skincare composition of claim 1, wherein the gold is in a form of a nanoparticle having a D50 particle size of no greater than about 100 nm.

6. The skincare composition of claim 1, wherein the metal-binding peptide comprises a copper-binding peptide.

7. The skincare composition of claim 1, wherein the metal-binding peptide comprises a copper heptapeptide-14 pantothenate.

8. The skincare composition of claim 1, wherein the lipid-modified peptide comprises heptapeptide-15 palmitate.

9. The skincare composition of claim 1, wherein each of the metal-binding peptide and the lipid-modified peptide comprises a heptapeptide.

10. The skincare composition of claim 1, wherein the carrier peptide comprises or is associated with a targeted delivery vehicle.

11. The skincare composition of claim 10, wherein the targeted delivery vehicle comprises at least one of lipid moiety or a metal-binding ligand.

12. The skincare composition of claim 1, wherein the signal peptide is included in the disclosed skincare composition in an amount of from about 0.00001 wt % to about 0.00005 wt %, based on a total weight of the skincare composition.

13. The skincare composition of claim 1, wherein the carrier peptide is included in the disclosed skincare composition in an amount of 0.00005 wt % to about 0.0002 wt %, based on a total weight of the skincare composition.

14. The skincare composition of claim 1, wherein the oat lipid composition comprises Avena sativa kernel extract, Avena sativa kernel oil, and Avena sativa kernel flour.

15. The skincare composition of claim 1, wherein the oat lipid composition is included in an amount within a range of about 1.0 wt % to about 4.0 wt %, based on a total weight of the skincare composition.

16. The skincare composition of claim 1, further comprising an evening primrose oil, in an amount within a range of about 0.5 wt % to about 1.0 wt %, based on a total weight of the skincare composition.

17. The skincare composition of claim 1, wherein the niacinamide is included in the disclosed skincare composition in an amount of about 1.0 wt % to about 4.0 wt %, based on a total weight of the skincare composition.

18. The skincare composition of claim 1, wherein the skincare composition is an aqueous composition.

19. The skincare composition of claim 1, wherein the skincare composition has a pH within a range of about 5.0 to about 6.0.

20. The skincare composition of claim 1, wherein the skincare composition has a viscosity within a range of about 8,000 cps to about 11,000 cps at 25° C.
